(19) United States
(12) Patent Application Publication
(10) Pub. No.: US 2026/0232943 A1
(43) Pub. Date: Aug. 13, 2026

(54) DEVICE AND METHOD OF PRODUCING HYPOXIC/HYPEROXIC/NORMOXIC GAS MIXTURES

(71) Applicant: Z+ HEALTH SRL, Noventa Vicentina (VI) (IT)

(72) Inventor: Antonio ZAMPERLA, Altavilla Vicentina (VI) (IT)

(21) Appl. No.: 19/160,644

(22) PCT Filed: Mar. 3, 2023
(86) PCT No.: PCT/EP2023/055497

§ 371 Date: Aug. 28, 2025

(51) Int. Cl. A61M 16/12; A61M 16/10; A61M 16/20

(52) U.S. Cl. CPC A61M 16/122; A61M 16/1005; A61M 16/105; A61M 16/202; A61M 2205/126; A61M 2205/3334; A61M 2205/7536

(57) ABSTRACT

FIELD: fire protection. SUBSTANCE: invention relates to fire-fighting equipment, namely to methods and devices for the prevention and exclusion of fire, fire containment with a limited supply of extinguishing agent in the form of a life-supporting hypoxic atmosphere controlled by signals from the danger zone. The gas separation device for creating a breathable fire-suppressing hypoxic atmosphere contains a working cabinet in which a gas distribution module is located, connected to a compressed air supply compressor with a pressure of 3-11 bar, equipped with a compressed air dryer and fine filters, while the gas separation module is made with the possibility of separating compressed air according to the degree of oxygen saturation into permeate-oxygen-enriched composition of the atmosphere and retentate-hypoxic composition, depleted in oxygen, with the possibility of its separation into a gas mixture, including: oxygen from 10% to 16%; inert gases; water vapor; carbon dioxide in quantities suitable for breathing with constant use as a fire-preventing atmosphere, or a gas mixture with an oxygen content of 9% to 12% for occasional use as a fire extinguishing agent. The gas separation device contains a gas separation module, including a hollow housing, in which a block of hollow-fiber tubular membranes is placed, consisting of a bundle of porous polymer fiber with a 10-100 nm thick gas separation layer applied to the outer surface, with the possibility of supplying a separated fraction: permeate of the separated gas into the inter-tube space and a hypoxic fraction: retentate in the form of oxygen-depleted air-into the outlet cavity of the gas distribution module. The block of hollow fiber membranes is hermetically fixed with the ends by means of epoxy collectors in the specified housing. The gas separation module is designed to operate in three modes of separation of the input air. The gas separation device is equipped with throttling means in the form of a system of pressure adjustment valves and oxygen content settings, and connected to a control panel equipped with oxygen sensors installed in protected rooms, it is equipped with a sensor and a temperature and humidity controller. EFFECT: increase in the fire protection of premises while maintaining the life support of the atmosphere. 6 cl, 2 dwg

### FIELD OF THE INVENTION

The present invention relates to a device and a method for producing hypoxic, hyperoxic and normoxic breathable gas mixtures. In particular, the device and the method according to the invention is suitable to provide a breathable gas mixture which complies with EU Pharmacopoeia, having a predefined amount of oxygen, that is selected and controlled depending on the final use of the gas mixture.

For example, the device and method according to the invention may be used to produce breathable gas mixtures that may be administered for a user's rehabilitation therapy, or may be used for providing intermittent hyperoxic/hypoxic training (IHHT) to a subject.

In other words, the present invention relates to a device and a method to produce breathable gas mixtures complying with EU Pharmacopoeia, having different oxygen concentrations.

### BACKGROUND OF THE INVENTION

It is known that breathing hypoxic air in the form of repeated short-term hypoxia exposures results in beneficial effects to the subject exposed.

Intermittent Hypoxic Training or Intermittent Hypoxic Treatment (IHT) is the common designation employed to indicate a treatment method using intermittent hypoxic exposure as the main therapeutic or training factor. A treatment session comprises a time interval breathing hypoxic (low oxygen) air from a dispensing device, for example a mask or canula, alternated with similar time intervals breathing ambient or hyperoxic air.

In case the alternation is between breathing hypoxic and ambient air, the designation of the treatment method is usually IHT (i.e. Intermittent Hypoxic Training or Treatment): in this case, a recovery time wherein a user removes the dispensing device, for example the mask, and breaths ambient air is then followed by another changeover to a low oxygen cycle.

In case the alternation is between breathing hypoxic and hyperoxic air, the designation of the treatment method is usually IHHT (i.e. Intermittent Hyperoxic/hypoxic Training or Treatment): in this case, a time interval wherein a user breaths hyperoxic air from the dispensing device is then followed by another changeover to a low oxygen cycle.

Standard practice is for the user to remain stationary while breathing hypoxic air via a hand-held mask. However, there is an interest in performing the treatment on an active subject, e.g. during physical exercise such as walking or running on a treadmill.

Known devices do not provide flows of gas mixture which allows IHT or IHHT on an active subject, e.g. during physical activity. IHT and IHHT deprive the body of oxygen in predetermined time intervals. During treatment, oxygen saturation levels in the blood, heart rate, and blood pressure are monitored in order to safely reduce oxygen levels.

The core of the IHT/IHHT method is repeated reduction of blood oxygen to the individual hypoxia adaptation level intermingled with recovery intervals. The intermittent exposure to hypoxia during IHT/IHHT stimulates adaptation to altitude, and results in better circulation, improved mitochondrial function, increased tolerance to toxic chemicals, increased antioxidant production and reduced inflammation.

In other words, hypoxia adaptation can enhance physical and mental capacity of an individual.

It should be noted that IHT/IHHT can be used for different application, including therapies and treatments used in rehabilitation for cardiovascular/pulmonary inquires, metabolic disorders and sport medicine; its use was also suggested in connection to dementia or Alzheimer's diseases.

IHT/IHHT can also be used in contexts other than rehabilitation or curative therapy, for example in preventive and anti-aging medicine concerning wellbeing, or for treating stressor-related disorders such as burnout and exhaustion, or sleep disturbances.

IHT/IHHT can also be used in sport and fitness training, since one of the effects of this training is an increased tolerance to physical load, comprising an increased \dot{V}O₂ max (i.e. increased maximal oxygen consumption, maximal oxygen uptake or maximal aerobic capacity) and increased exercise-until-exhaustion (ETE) time.

Alternating hypoxic air with hyperoxic air, or Intermittent Hypoxic Hyperoxic Treatment (IHHT) is also known in the art.

US2009183738 discloses a device and a method for providing alternated hypoxic and hyperoxic air mixtures to be supplied to a user for intermittent breathing the said mixtures in order to improve cognitive and functional abilities of the said user.

According to US '738 breathable air mixtures for an IHHT are generated with a low concentration of oxygen (1-18%) or with a high concentration of oxygen in (25-45%), by separation of ambient air, and then fed to the user via a face mask.

A problem of known methods for generating breathable air mixtures is that the devices supply permeate (hyperoxic) flow and retentate (hypoxic) flow having different flow rates, in particular a hypoxic flow rate which is significantly greater than the hyperoxic flow rate.

Another problem of the known devices and methods is the reduced life duration of device using pressure swing adsorption (PSA) technology.

Another disadvantage is that the PSA process requires a stabilisation time on start-up. This can vary from several seconds to hours, depending on the design of the system. Response to changes of oxygen requirements by the user in known art may be too slow.

It is therefore an aim of the present invention to solve the above problems and provide a device and a method for producing breathable air mixtures having different concentrations of oxygen, where short times for changing the gas mixture composition are possible. Apparatus and method are particularly useful for an intermittent hypoxic/normoxic/hyperoxic treatment.

Another aim of the present invention is to provide a device for producing breathable gas mixtures having a longer duration with respect to known device.

Said aims and others, are reached by the present invention that provides a device according to claim 1 and a method according to claim 12. Preferred embodiments are recited in dependent claims.

### SUMMARY OF THE INVENTION

The present solution provides a device for producing hypoxic (mode A), hyperoxic (mode B) and normoxic (mode C) breathable gas mixtures comprising:
at least one source of pressurized air for feeding pressurized air to the air supply line,
at least one membrane tube assembly for separating air into a retentate hypoxic gas mixture and a permeate hyperoxic gas mixture, comprising a pressurized air inlet, a hypoxic outlet fluidly connected to said hypoxic output channel comprising valve means, and a hyperoxic outlet fluidly connected to said hyperoxic output channel comprising valve means,
means for supplying a breathable gas mixture, preferably complying with EU Pharmacopoeia, positioned along said air supply line upstream of said membrane tube assembly, and comprising at least one air dryer module and at least one filtration unit, and
means for controlling the pressure of the pressurized breathable gas mixture, positioned along a pressure regulation branch downstream of said means for supplying breathable gas mixtures and upstream of said membrane tube assembly, and fluidly connected through valve means with the pressurized air inlet of said membrane tube assembly and with the normoxic output channel,
a control unit adapted to store operating parameters of said device according to said hypoxic, hyperoxic and normoxic modes, connected at least to said means for controlling the pressure of the pressurized breathable gas mixture, and connected at least to said valve means for controlling the flow of hypoxic, hyperoxic or normoxic gas mixtures through the respective hypoxic, hyperoxic or normoxic output channels.

The device according to the invention is advantageously adapted to supply hypoxic, hyperoxic and normoxic breathable gas mixtures one after the other in a required sequence.

It has to be noted that the gas delivery from the device is almost instantaneous for the normoxic mixture, and also for the hypoxic and hyperoxic mixtures, since the membrane tube assembly delivers the gas mixture with the predefined oxygen content as soon as it reaches a suitable operating pressure. In this way, a user can be subjected to a normoxic cycle between a hyperoxic cycle and a hypoxic cycle, and does not have to remove the breathing device, such as the mask, in order to breath normoxic ambient air.

A further advantage is that the air stream which is fed to the membrane tube assembly is already dry, as well as CO and CO₂ contaminants relieved, since it is a breathable gas mixture complying with the requirements of EU Pharmacopoeia.

Indeed, the breathable gas mixtures produced with the described device comply with the EU Pharmacopoeia, preferably with the monography n° 1238 “Medicinal Air” (Aer Medicinalis) of the European Pharmacopoeia, 11^{th} edition, in terms of CO, CO₂, H₂O and oil residual content.

According to an aspect, the device comprises a dispensing channel and a dispensing device for a user, for delivering said hypoxic, hyperoxic and normoxic breathable gas mixtures.

According to an aspect, the hypoxic gas mixture comprises an oxygen percentage comprised between 13%±1%-15%±1%, preferably of 14%±1%, the hyperoxic gas mixture comprises an oxygen percentage comprised between 34%±1%-36%±1%, preferably of 35%±1%, and the normoxic gas mixture comprises an oxygen percentage comprised between 20%±1%-22%±1%, preferably of 21%±1%.

According to an aspect the said means for controlling the pressure of the pressurized breathable gas mixture are fluidly connected to said at least one source of pressurized air through a recirculation duct, for feeding back at least a portion of the pressurized breathable gas mixture to said source of pressurized air.

Advantageously, as a matter of fact, the air stream which is feedback through the recirculation duct is already dry, as well as oil, CO and CO₂ contaminants relieved, since it is the breathable gas mixture which is supplied by the means for supplying a breathable gas mixture according to EU Pharmacopoeia, thus giving a less burdensome load to the line filters and air dryer module.

According to an aspect, the control unit is configured to operate said means for controlling the pressure such that:
in said hypoxic mode the air pressure value is comprised between 5 bar (g) and 6 bar (g), preferably equal to 5.5 bar (g);

in said hyperoxic mode and in said normoxic mode the air pressure value is comprised between 8 bar (g) and 9 bar (g), preferably equal to 8.5 bar (g).

It has to be noted that the above mentioned pressure values are maintained according to a possible embodiment of the invention, in order to exploit a desired response flow rate. Anyway, possible embodiments wherein different pressure values are provided, in order to exploit different response flow rates are not excluded.

Advantageously, the response flow rates obtained with such pressure values are useful for IHT/IHHTprotocols in sport and fitness training for increasing tolerance to physical load, comprising an increased \dot{V}O₂ max (i.e. increased maximal oxygen consumption, maximal oxygen uptake or maximal aerobic capacity) and increased exercise-until-exhaustion (ETE) time.

Advantageously, in the hypoxic and hyperoxic modes A, B, the pressurized breathable air is supplied to the membrane tube assembly, which is commanded through proper value of the air pressure and/or of the flow rate, such that the desired composition of the permeate and/or retentate gas mixtures may be obtained, namely:
a feed air pressure set at value comprised between 5 bar (g) and 6 bar (g), preferably equal to 5.5 bar (g) for the hypoxic mode “A” is suitable for exploiting a response flow rate of ~30 l/min of hypoxic air stream through the hypoxic outlet depleted of a value comprised between 13%±1%-15%±1%, preferably 14%±1% of O₂ titre (concentrate=retentate side flowing under pressure),
a feed air pressure set at value comprised between 8 bar (g) and 9 bar (g), preferably equal to 8.5 bar (g), for the mode “B” is suitable for collecting a response flow rate of ~23 l/min of hyperoxic air stream through the hyperoxic outlet enriched by 34%±1%-36%±1%, preferably 35%±1% O₂ titre concentrated in said air (permeate side naturally flowing at atmospheric pressure).

In the normoxic mode C, the feed air pressure can be set by the means for controlling the pressure Pi at a value comprised between 8 bar (g) and 9 bar (g), preferably equal to 8.5 bar (g) for collecting a flow rate of ~35 l/min of normoxic air with 20%±1%-22%±1%, preferably 21%±1% nominal O₂ titre.

According to an aspect, the air dryer module comprises a regenerative inlet fluidly connected through at least one hypoxic regenerative channel with said hypoxic output channel.

According to an aspect, the air dryer module comprises a regenerative inlet fluidly connected through at least one normoxic regenerative channel with said normoxic output channel.

More in particular, according to this aspect, the at least one hypoxic regenerative channel comprises valve means which can be operated for controlling, preferably for alternatively stopping or allowing, a flow of hypoxic gas mixtures to said regenerative inlet of said air dryer module in said hypoxic mode or hyperoxic mode, and wherein said normoxic regenerative channel comprises valve means which can be operated for controlling, preferably for alternatively stopping or allowing, the flow of normoxic gas mixtures to said regenerative inlet of said air dryer module in said normoxic mode.

In this way, the air dryer module is regenerated and cooled, and its life duration is enhanced.

Furthermore, when serving the hyperoxic operating mode, the retentate which would be considered as a by-product, is not wasted, i.e. discharged, but is used for regeneration of the air dryer module.

According to an aspect, the device comprises means for controlling the flow rate of the breathable gas mixture provided by said device, positioned along said dispensing channel upstream of said dispensing device.

As above mentioned, the device comprise means for filtering and drying the inlet air, i.e. the at least one filtration unit and the dryer module, such that the produced breathable gas mixtures comply with the EU Pharmacopoeia, preferably with the monography n° 1238 “Medicinal Air” (Aer Medicinalis) of the European Pharmacopoeia, 11th edition, in terms of CO, CO₂, H₂O and oil residual content. In particular, according to an aspect, the at least one filtration unit for filtering the air supplied by the source of pressurized air comprises a filter cartridge for reducing the value of the carbon dioxide (CO₂) contaminant in said air below 500 ppm, and/or a filter cartridge for reducing the carbon monoxide (CO) contaminant in said air below 5 ppm, and/or a coalescer filter for capturing H₂O droplets and for removing solid dust particles having an average dimension of at least 0.1 μm, and/or a dust filter for removing solid dust particles having an average dimension of at least 0.1 μm from said air.

According to an aspect, the hyperoxic output channel comprises a venting branch for venting the permeate hyperoxic gas mixture when the device is operated in said hypoxic mode. The permeate is discharged at a distance from the air compressor suction port to avoid it being sucked into the compressor.

The present invention is further directed to a method for operating a device according to the invention, for providing hypoxic, hyperoxic and normoxic breathable gas mixtures, wherein said method comprises the following steps:
(a) Selecting the operating mode of the device among the hypoxic mode, hyperoxic mode and normoxic mode stored in the control unit,
(b) feeding pressurized air to the means for providing a flow of breathable gas mixture to the pressure regulation branch;
(c) operating the means for controlling the pressure of the breathable gas mixture along the pressure regulation branch so as to maintain an output pressure level set by the control unit according to the selected mode of operation,
(d) feeding the pressurized breathable gas mixture to the pressurized air inlet of the membrane tube assembly in the hypoxic or hyperoxic modes of operation of the device, or to the normoxic output channel in the normoxic mode of operation of the device, wherein
in the hypoxic mode a hypoxic gas mixture is supplied by the membrane tube assembly through the hypoxic outlet, to the hypoxic channel, and to the dispensing device for a user,
in the hyperoxic mode a hyperoxic gas mixture is supplied by the membrane tube assembly through the hyperoxic outlet, to the hyperoxic channel, and to the dispensing device for a user
in the normoxic mode, a normoxic gas mixture is supplied by the means for controlling the pressure to the normoxic channel, and to the dispensing device for a user.

According to an aspect, at least a portion of the pressurized breathable gas mixture is recirculated back through a recirculation duct to the source of pressurized air, in order to reduce the pressure value of the pressurized breathable gas mixture according to the selected mode of operation of the device.

Advantageously, this step allows to reduce the pressure value of the breathable gas mixture, according to the selected mode of operation of the device, while recirculating a portion of the breathable gas mixture back to the source of pressurized air.

In this way, the air which is fed to the device by the source of pressurized air, is at least partially already deprived by contaminant particles, thus giving a less burdensome load to the line filters and air dryer module.

According to an aspect, in said hypoxic mode the means for controlling the pressure of the breathable gas mixture are operated for providing an outlet pressure value comprised between 5 bar (g) and 6 bar (g), preferably equal to 5.5 bar (g);
in said hyperoxic mode and in said normoxic mode the means for controlling the pressure of the breathable gas mixture are operated for providing an air pressure value comprised between 8 bar (g) and 9 bar (g), preferably equal to 8.5 bar (g).

According to an aspect, in said hypoxic mode, the hypoxic gas mixture supplied by the membrane tube assembly comprises an oxygen percentage comprised between 13%±1%-15%±1%, preferably of 14%±1%, and in said hyperoxic mode the hyperoxic gas mixture supplied by the membrane tube assembly comprises an oxygen percentage comprised between 34%±1%-36%±1%, preferably of 35%±1%, and
in said normoxic mode, the normoxic gas mixture supplied by the means for controlling the pressure of the breathable gas mixture comprises an oxygen percentage comprised between 20%±1%-22%±1%, preferably of 21%±1%.

According to an aspect, the method comprises the step of feeding a flow of hypoxic gas mixture to the air dryer module.

According to an aspect, the method comprises the step of feeding a flow of normoxic gas mixtures to the air dryer module.

### DESCRIPTION OF THE FIGURES

Exemplary embodiments of the present invention are now described in greater detail with reference to the accompanying drawings provided by way of non-limiting example, wherein:

FIG. 1 is a block diagram of a possible embodiment of the device 1 according to the invention, operating in hypoxic mode;

FIG. 2 is a block diagram of a possible embodiment of the device 1 according to the invention, operating in hyperoxic mode;

FIG. 3 isa block diagram of a possible embodiment of the device 1 according to the invention, operating in normoxic mode;

FIG. 4 is a chart showing the flows of the hypoxic and hyperoxic gas mixtures produced with the system according to the invention.

FIG. 5 is a schematic view of an individual hollow fibre membrane according used in the invention;

FIG. 6 is a schematic view of the membrane tube assembly 2 according to the invention.

### DETAILED DESCRIPTION OF THE INVENTION

The present invention describes, with reference to the related figures, a device 1 for producing hypoxic, hyperoxic and normoxic breathable gas mixtures, which can be alternatively operated according to, i.e. switched between, at least three operating modes A, B, C, for alternatively providing hypoxic (mode A), hyperoxic (mode B) and normoxic (mode C) breathable gas mixtures, through respective hypoxic, hyperoxic and normoxic output channels 30′, 30″, 30″′.

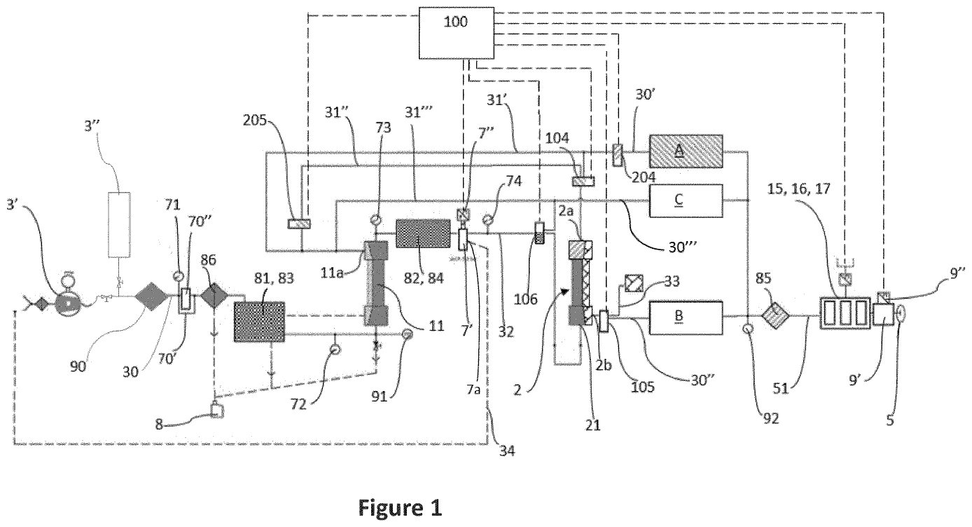

With reference to FIG. 1, the respective hypoxic, hyperoxic and normoxic output channels 30′, 30″, 30′″ are fluidly connected through a dispensing channel 51 to a dispensing device 5 for a user.

The device 1 according to the invention comprises a control unit 100 adapted to store the operating parameters of said hypoxic, hyperoxic and normoxic modes A, B, C.

As will be further disclosed in the following, according to a possible embodiment, the relevant parameters of the hypoxic, hyperoxic and normoxic modes A, B, C stored in the control unit 100 comprise at least the pressure value Pi of the gas mixture and/or the flow rate F of the gas mixture, and/or the temperature T of the gas mixture, and/or the Oxygen content of the gas mixture. Preferably all the said parameters are stored, i.e. memorized in the control unit 100.

According to a possible embodiment, the dispensing device 5 comprises one of a nasal cannula or a face mask.

In a possible embodiment, the device 1 comprises at least one delivery gas breathing bag fluidly connected with said dispensing channel 51, wherein the gas mixtures can be stored before being delivered to the user.

The delivery gas breathing bag can further comprise a level/volume sensor connected to control unit 100, for monitoring the volume of the mixture inside the breathing bag.

With reference to FIGS. 1-3, each output channel 30′, 30″, 30′″ comprises respective valve means 104, 105, 106 controlled and operated by the control unit 100 to control, and preferably to alternatively allow or prevent the flow of hypoxic, hyperoxic or normoxic gas mixtures through the respective hypoxic, hyperoxic or normoxic output channels 30′, 30″, 30′″.

According to a preferred embodiment, the valve means 104, 105, 106 may be solenoid valves.

The device 1 is provided with an air supply line 30, and at least one source of pressurized air 3′, 3″ feeds pressurized air to the air supply line 30. In a possible embodiment, the at least one source of pressurized air is a compressor 3′, such as a fixed speed compressor 3′.

A suitable working pressure is preferably in the range of 9.5 bar (g) to 10.0 bar (g).

In a further possible embodiment, the source of pressurized air 3 provides air from an externally sourced compressed air stream 3″.

By this last alternative, the feed air pressure is preferably in the range of 9.5 bar (g) to 10.0 bar (g) and the rated flow is to be no less than 70 NI/min.

At least one membrane tube assembly 2 is provided in the device 1 according to the invention, for separating air, in particular the pressurized air supplied by the at least one source of pressurized air 3′, 3″, into a retentate hypoxic gas mixture and a permeate hyperoxic gas mixture.

According to a possible embodiment shown in FIG. 6, the membrane tube assembly 2 comprises individual hollow fibres 200, and it is capable of separating air components, thus producing gas mixtures in the form of oxygen enriched air at the hyperoxic outlet 2b and oxygen depleted air at the hypoxic outlet 2a.

Suitable membrane tube assemblies are commercially available, e.g. as gas separators from Evonik, under the name SEPURAN® NG.

In particular, the membrane tube assembly 2, is a so called N₂/O₂ selective membrane separator, which comprises bundles of hollow fibres 200 contained within a tube assembly 2. In a preferred embodiment, the hollow fibres 200 have a permeable membrane that selectively separate pressurized air into a low oxygen mixture (retentate) at the hypoxic outlet 2a and an oxygen enriched mixture (permeate) at the hyperoxic outlet 2b, and an outer tube which provides the structural strength to membrane tube assembly 2.

The hollow fibres 200 consist, in a way known in the art, of a permeable structure with an ultrathin cover layer, wherein the hollow fibres are held in a module which is a metal or plastic tube.

Preferably, the ends of fibres are bound, e.g. glued, together and are fixed to the tube so that pressurized air fed to the air inlet 21, enters the fibres.

The membrane fibres allow oxygen and water molecules of the pressurized air to permeate through the wall of the membrane fibers in the membrane tube assembly 2 faster than the nitrogen molecules, such that most of water and oxygen molecules are discharged from the membrane tube assembly as a permeate product, through the hyperoxic outlet 2b.

With reference to FIGS. 1-3 , the membrane tube assembly 2 comprises a pressurized air inlet 21, a hypoxic outlet 2a which is fluidly connected to the hypoxic output channel 30′, and a hyperoxic outlet 2b fluidly connected to the hyperoxic output channel 30″.

In a preferred embodiment shown in FIG. 1-3 , the hyperoxic and hypoxic output channels 30′, 30″ comprise respective valve means 104, 105.

The device 1 according to the invention comprises means 81, 82, 83, 84; 11 for supplying a breathable gas mixture positioned along the air supply line 30 upstream of said membrane tube assembly 2, and comprising at least one air dryer module 11 and at least one filtration unit 81, 82, 83, 84 positioned upstream of the membrane tube assembly.

In order to produce a breathable gas mixture complying with the EU Pharmacopoeia, and in particular complying with the residual contaminant value prescribed by the EU Pharmacopoeia, according to a possible embodiment the at least one filtration unit 81, 82, 83, 84 for filtering the air drawn by the source of pressurized air 3′, 3″ comprises one or more of the following filter types:
a filter cartridge 81 for reducing the value of the carbon dioxide CO₂ contaminant present in the drawn air below 500 ppm.

For example, the filter 81 is a filter cartridge type filled with soda lime for sorbing the carbon dioxide (CO₂) contaminant.
a filter cartridge 82 for reducing the carbon monoxide CO contaminant present in the drawn air below 5 ppm.

For example, the filter 82 is a filter cartridge type filled with hopcalite catalytic product to reduce that CO contaminant to a residual value as prescribed by the “Aer medicinalis” of the European Pharmacopoeia, i.e. <5 ppm.
a coalescer filter 83 for capturing H₂O droplets and for removing solid dust particles having an average dimension of at least 0.1 μm from the drawn air. In particular, a coalescer filter is used for capturing the droplets of H₂O generated in previous filter cartridge 81 and for removing 99.9+% of solid dust particles having an average dimension of 0.1 μm.
a dust filter 84 for removing solid dust particles having an average dimension of at least 0.1 μm from the drawn air, in particular the solid dust particles which could be carried over from filter cartridge 82 filled with hopcalite catalytic product.

It has to be noted that, according to possible embodiments, the device 1 can comprise other types of filters, for example a bacterial filter 85 positioned along the dispending channel 51 for capturing possible bacteria and virus, and/or a moisture separator 86, positioned downstream the pressurized air source 3′, 3″ and upstream the means 81, 82, 83, 84; 11 in order to reduce the water vapour value before the filter chain.

According to a possible embodiment, the air dryer module 11 allows a non-condensing feed-air supply for the membrane tube assembly 2.

The dryer/evaporation module 11 is suitable to lower the dewpoint of the compressed air stream fed to the membrane tube assembly 2 such that the pressure dewpoint of the compressed air needs is at least 5° C. lower than the lowest ambient temperature to be expected, and remove the condensate that is formed.

According to a possible embodiment, the liquid condensate coming from moisture separator 86 or coalescer filter 83 and by the air dryer module 11 is fed to a collecting canister 8.

Preferably, with reference to FIGS. 1-3 , the dryer/evaporation module 11 is positioned along the air feeding line 30, downstream of the source of pressurized air 3 and upstream of the membrane tube assembly 2, preferably between the couple consisting of filter cartridge 81 and coalescer filter 83 and a second couple of filter cartridge 82 and a dust filter 84 for solid dust particles. The device 1 according to the invention comprises means 7′, 7″ for controlling the pressure Pi of the pressurized breathable gas mixture, positioned along a pressure regulation branch 32 downstream of said means 81, 82, 83, 84; 11 and upstream of said membrane tube assembly 2.

The means 7′, 7″ for controlling the pressure Pi are fluidly connected through valve means 106 positioned along the pressure regulation branch 32, with the pressurized air inlet 21 of the membrane tube assembly 2 and with the normoxic output channel 30″′.

With reference to FIG. 1-3, the means 7′, 7″ for controlling the pressure Pi of the breathable gas mixture comprise a valve 7′, preferably a proportional solenoid valve 7′, for maintaining a preset value of the output pressure level on the pressure regulation branch 32 (according to the parameters of the operating modes A, B, C stored in the control unit 100), and a pressure transducer 7″ which provides a pressure feedback signal to the control unit 100.

It has to be noted that the means 7′, 7″ for controlling the pressure Pi of the pressurized breathable gas mixture operate a back pressure regulation in a way known per se in the art.

Back pressure regulators are devices well known in the art.

It has to be noted that Back Pressure Regulators (BPR) are known in the art as devices that maintain a defined pressure upstream of their own inlet. When the fluid pressure at the inlet of the back pressure regulator exceeds a predetermined set point, the regulator opens to relieve the excess pressure. According to a possible embodiment, the means 7′, 7″ for controlling the pressure Pᵢ of the pressurized breathable gas mixture are fluidly connected to the at least one source of pressurized air 3′, 3″ through a recirculation duct 34, for recirculating back at least a portion of the pressurized breathable gas mixture to said source of pressurized air 3′, 3″.

As will be better discussed below, at least a portion of the pressurized breathable gas mixture is recirculated back to the source of pressurized air 3′, 3″, through the recirculation duct 34, in order to reduce the pressure value Pi of the breathable gas mixture, according to the selected operating mode A, B, C of the device 1: when the pressure value of the breathable gas mixture at the inlet of the means 7′, 7″ for controlling the pressure exceeds a predetermined set point stored in the control unit 100, the regulator opens to relieve the excess pressure through the recirculation duct 34.

According to a possible embodiment, the device 1 can comprise further pressure regulation means 70′, 70″, positioned downstream of the pressurized air source 3′, 3″ and upstream of the means 81, 82, 83, 84; 11.

The further pressure regulation means 70′, 70″ are useful for smoothly priming the pressure build-up when the device 1 is commanded to start operating.

The further pressure regulation means 70′, 70″ can comprise a fix orifice restriction 70′ for protecting the downstream air dryer module 11 and membrane tube assembly 2 from harmful hammering due to pressure variations.

A normally closed valve 70″, preferably a normally closed solenoid valve 70″ then opens to full downstream flow upon a time delay, i.e. just after the preceding fix orifice restriction 70′ has terminated the initial but smooth pressure build-up of the system.

According to a possible embodiment, the device 1 comprises means 9′, 9″ for controlling the flow rate F of the breathable gas mixture, positioned along the dispensing channel 51 upstream of the dispensing device 5.

The pressure Pi, the temperature T and the flow rate F at which the compressed air enters the fibres of the membrane tube assembly 2, as well as the time the air remains inside the membrane tube assembly 2, determines the oxygen content in the hyperoxic and hypoxic gas mixtures at the hypoxic and hyperoxic outlets 2a, 2b.

In particular, in a possible embodiment according to the invention, the membrane tube assembly 2 operates at air inlet pressures Pᵢ ranging from 5 bar (g) to 10 bar (g).

The amount of hypoxic gas mixtures that can be produced at the outlet 2a of the membrane tube assembly 2 increases proportionally with the value of the air inlet pressure Pᵢ.

As above mentioned, the performance of the tube membrane 2 in terms of oxygen percentage in the hyperoxic/hypoxic gas mixtures, depends on several parameters, among which the pressure value Pᵢ of the pressurized air at the inlet 21 of the tube membrane 2, the flow rate F of the gas mixture at the dispensing channel 51, and the temperature T of the pressurized air fed at the inlet 21 of the tube membrane 2.

In more detail, by varying the flow rate F of the retentate gas mixture at the dispensing channel 51, the oxygen content in the hyperoxic/hypoxic gas mixtures at the respective outlets 2b, 2a will change.

In particular, with decreasing the flow rate F of the gas mixture at the dispensing channel 51, the residence time of the air in the tube membrane 2 will increase and as a result the oxygen content in the hypoxic gas mixture at the second outlet 2a will be lowered and the oxygen content in the hyperoxic gas mixture at the first outlet 2b will be increased.

For example, the percentage content of oxygen in the hyperoxic/hypoxic mixtures can be adjusted by tuning the flow rate F of the retentate gas mixture at the dispensing channel 51.

The flow rate, pressure and preferably also temperature are adjusted and set according to the required parameters of retentate and permeate gas mixtures in a way know per se in the art. In fact, producers of the membrane tubes usually provide relevant charts showing changes of composition as a function of at least the flow rate and pressure.

With reference to FIGS. 1-3, the device 1 according to the invention comprises a control unit 100 adapted to store operating parameters of the device 1 according to the hypoxic, hyperoxic and normoxic modes A, B, C, connected at least to the means 7′, 7″ for controlling the pressure Pi of the pressurized breathable gas mixture fed to the membrane tube assembly 2, or to the normoxic output channel 30″′.

Furthermore, the control unit 100 is connected at least to the valve means 104, 105, 106 for controlling the flow of hypoxic, hyperoxic or normoxic gas mixtures through the respective hypoxic, hyperoxic or normoxic output channels 30′, 30″, 30″′.

In a possible embodiment, the control unit 100 can be further connected to said means 9′, 9″ for controlling the flow rate of the breathable gas mixture.

With reference to FIG. 1-3, in a possible embodiment, the device 1 further comprises at least one, preferably a plurality of pressure sensors 71, 72, 73, 74 preferably positioned at different relevant positions along the pressurized air feed line 30.

The pressure sensors 71, 72, 73, 74 can be connected to the control unit 100, for monitoring the current value of the pressure Pi of the pressurized air.

The performance of the tube membrane 2 is influenced by the temperature T at which the membrane tube assembly 2 operates: the membrane tube assembly 2 operates optimally at a temperature T comprised in the range of 2° C. to 50° C. and increasing temperature will result in higher pressurized air consumption.

For this reason, the device 1 comprises means 90, 91, 92 for monitoring the temperature T of the pressurized air along the air supply line 30, which may comprise an air cooler 90.

Preferably, according to a possible embodiment, the air cooler 90 is an aftercooler 90 positioned upstream the means 81, 82, 83, 84; 11 for supplying a breathable gas mixture. As known in the art an aftercooler 90 is a mechanical heat exchanger designed to remove the heat and moisture of compression from a compressed air stream so the air is cool and dry enough for use in the device 1, thus giving a less burdensome load to the line filters 81, 82, 83, 84 and air dryer module 11.

A compressed air aftercooler 90 has the primary functions of cooling the air discharged from the air compressor 3′, 3″, reducing the moisture in compressed air, protecting downstream line filters 81, 82, 83, 84 and air dryer module 11 from excessive heat and moisture.

In a possible embodiment, said means for controlling the temperature T of the pressurized air along the air supply line 30, further comprise at least one temperature sensor 91, for detecting the temperature of the air fed from the source of pressurized air 3′, 3″ to the air dryer module 11. Preferably, the temperature sensor 91 is connected to the electronic control unit 100.

It has to be noted that the temperature sensor 91 is useful for monitoring the temperature entering the succeeding air dryer module 11. Since the performance of the device 1 is quite dependent of the isotherm feeding the membrane tube assembly 2 (the lower the temperature is, the better performance results steady), the expected set point is comprised between 0° C. and 50° C., and preferably should not exceed the value of 40° C.

Furthermore, as above mentioned, in a possible embodiment, said means for controlling the temperature T of the pressurized air along the air supply line 30, further comprise at least one temperature sensor 92, for detecting the temperature of the air fed to dispensing channel 51. Preferably, the temperature sensor 92 is connected to the electronic control unit 100.

According to a possible embodiment, the air dryer module 11 comprises a regenerative inlet 11a fluidly connected through at least one hypoxic regenerative channel 31′, 31″ with the hypoxic output channel 30′.

According to a further possible embodiment, the regenerative inlet 11a is fluidly connected through a normoxic regenerative channel 31″′ with the normoxic output channel 30″′.

It has to be noted that, when the device 1 is operated under hypoxic or hyperoxic mode A, B, at least a portion of retentate hypoxic flow, leaving the membrane tube assembly separator 2 is used as sweep fluid exploited for regenerating the air dryer module 11.

In other words, in said hypoxic mode A, shown by way of example in FIG. 1, at least a portion of retentate hypoxic flow provided by the membrane tube assembly 2 through the hypoxic output channel 30′, is supplied over to the air dryer module 11 for its regeneration purpose through a first hypoxic regenerative channel 31′.

It has to be noted that, according to a possible embodiment, the hyperoxic output channel 30″ comprises a venting branch 33, for venting hyperoxic gas mixture when the device 1 is operated in said hypoxic mode A.

In said hyperoxic mode B at least a portion of retentate hypoxic flow provided by the membrane tube assembly 2 through the hypoxic output channel 30′, is supplied over to the air dryer module 11 for its regeneration purpose through a second hypoxic regenerative channel 31″.

When in operation in said hypoxic mode A, the valve means 104 of said hypoxic output channel 30′ fluidly connects the hypoxic outlet 2a of the membrane tube assembly 2 with both the dispensing device 5 and the hypoxic regenerative channel 31′: in this hypoxic mode A, a main portion of the hypoxic gas mixture flowing from the membrane tube assembly 2 is supplied to the dispensing device 5, while a portion of the hypoxic air is supplied to the air dryer module 11 for regeneration purpose.

On the other side, during this hypoxic mode A, the valve means 105 of the hyperoxic output channel 30″ connects the hyperoxic outlet 2b of the membrane tube assembly 2 with a venting branch 33, which discharges the hyperoxic gas mixture to the atmosphere.

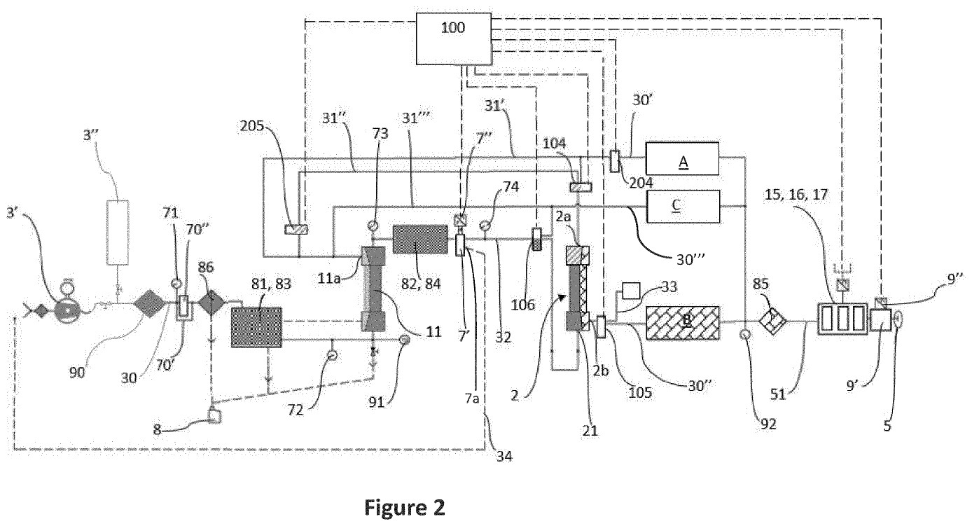

When in operation in said hyperoxic mode B, shown by way of example in FIG. 2, the valve means 105 of said hyperoxic output channel 30″ fluidly connects the hyperoxic outlet 2b of the membrane tube assembly 2 with the dispensing device 5, while the valve means 104 of the hypoxic output channel 30′ connects the respective hypoxic outlet 2a of the membrane tube assembly 2 with the second hypoxic regenerative channel 31″, which supplies hypoxic gas mixture to the regenerative inlet 11a of the air dryer module 11.

In this way, the hypoxic or the hyperoxic gas mixtures are alternatively fed to the dispensing device 5, while a portion of the hypoxic gas mixture is always provided to the air dryer module 11 for regeneration purpose in both the hypoxic or hyperoxic mode A, B. In this way the device 1 can be used for intermittent hyperoxic/hypoxic training (IHHT).

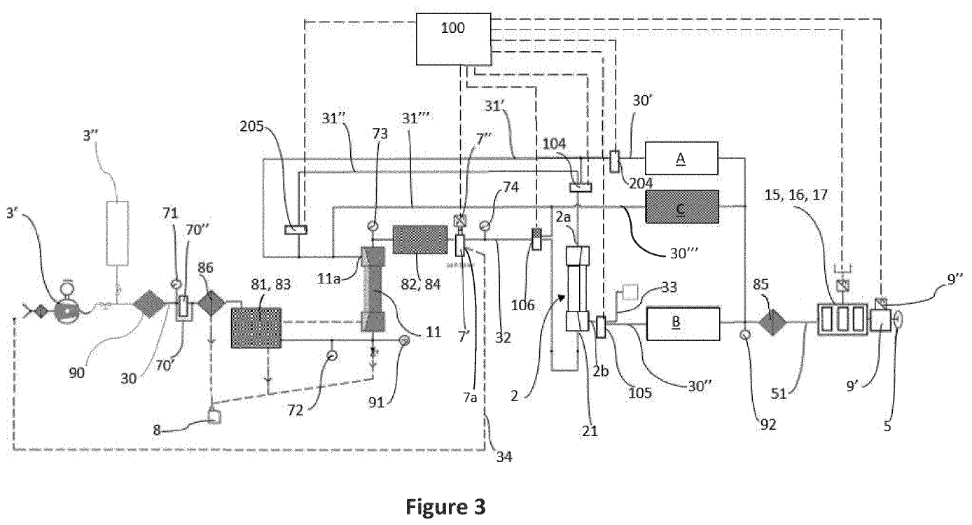

Conversely, when the device 1 is operated under normoxic mode C, shown by way of example in FIG. 3, at least a portion of normoxic gas mixture, leaving the means 7′, 7″ for controlling the pressure Pi is used as sweep fluid exploited for regenerating the air dryer module 11.

In other words, in said normoxic mode C, a main portion of normoxic gas mixture provided by the means 7′, 7″ for controlling the pressure Pi through the normoxic output channel 30″′, is supplied to the dispensing device 5, while at least a portion of normoxic gas mixture provided by the means 7′, 7″ for controlling the pressure Pi, is supplied over to the air dryer module 11 for its regeneration purpose.

According to a possible embodiment, the at least one hypoxic regenerative channel 31′, 31″ comprises valve means 204, 205 which can be operated for controlling, and preferably for avoiding or allowing, the flow of hypoxic gas mixtures to the regenerative inlet 11a of the air dryer module 11 in the hypoxic mode A or hyperoxic mode B.

Similarly, in the normoxic mode C, show in FIG. 3 the normoxic regenerative channel 31″′ comprises valve means which can be operated for controlling, and preferably for alternatively avoiding or allowing, the flow of normoxic gas mixtures to the regenerative inlet 11a of the air dryer module 11.

The valve means 204, 205, positioned along respective hypoxic regenerative channel 31′, 31″as shown in FIG. 1-3 and the valve means positioned along the normoxic regenerative channel 31″′ are connected to, and controlled by the control unit 100.

With reference to FIG. 1-3, at least one transducer 15, 16, 17 is positioned along the dispensing channel 51 and connected to the control unit 100 for the purpose of controlling and operating the valve means 204, 205 of the at least one hypoxic regenerative channel 31′, 31″.

As above discussed, the device 1 can be alternatively operated according to three different modes, for alternatively providing hypoxic, hyperoxic or normoxic gas mixtures.

To this purpose, according to a possible embodiment, the control unit 100 is configured to operate the means 7′, 7″ for controlling the pressure Pi for obtaining different pressure values.

It has to be noted that in the present description the pressure value is given in gauge pressure (bar g), i.e. pressure in bars above ambient or atmospheric pressure (1 bar):
in said hypoxic mode A the control unit 100 is configured to operate the means 7′, 7″ for controlling the pressure Pi such that the air pressure value is comprised between 5 bar (g) and 6 bar (g), preferably equal to 5.5 bar (g); in said hyperoxic mode B and in said normoxic mode C control unit 100 is configured to operate the means 7′, 7″ for controlling the pressure Pi such that the air pressure value is comprised between 8 bar (g) and 9 bar (g), preferably equal to 8.5 bar (g).

As above discussed, the means 7′, 7″ for controlling the pressure Pi comprise a valve 7′, preferably a proportional solenoid valve 7′, for maintaining a presetted output pressure level on the pressure regulation branch 32, and a pressure transducer 7″ for providing a feedback signal.

With reference to FIGS. 1-3, the device 1 comprise a recirculation duct 34, which fluidly connects an outlet port 7a of the valve 7′ with the at least one source of compressed air 3′.

In this way, according to the selected mode of operation A, B, C of the device 1, the excess of pressure of the pressurized breathable gas mixture (supplied by the means 81, 82, 83, 84; 11 for supplying a breathable gas mixture having residual contaminants value complying with EU Pharmacopoeia) is modulated and relieved by the means 7′, 7″ for controlling the pressure Pi, by allowing the recirculation of a portion of the breathable gas mixture through the recirculation branch 34, in order to obtain different pressure values according to the selected operating mode A, B, C.

In particular, according to a preferred embodiment, the excess of pressure of the pressurized air fed by the source of pressurized air 3′, 3″ is relieved by the outlet port 7a of the proportional solenoid valve 7′, modulated by the feedback signal provided by the pressure transducer 7″.

In fact, in case the source of pressurized air is a fix speed compressor 3′, supplying pressurized air having a pressure value comprised between 9.5 bar (g) and 10 bar (g), for all the three modes A, B, C there is an excess of air pressure value, and a portion of air is recirculated back to the inlet of the device 1, for compressor 3′ recirculation, through a recirculation duct 34.

As a matter of fact, the air stream which is recirculated back through the recirculation duct 34 is already dry, as well as oil, CO and CO₂ contaminants relieved, since it is the breathable gas mixture which is supplied by the means 81, 82, 83, 84; 11 for supplying a breathable gas mixture having residual contaminants values complying with EU Pharmacopoeia, thus giving a less burdensome load to the line filters 81, 82, 83, 84 and air dryer module 11.

The breathable air, whose pressure value is regulated and controlled by the means 7′, 7″ for controlling the pressure Pi, is provided through valve means 106, to the pressurized air inlet 21 of the membrane tube assembly 2 (in said hypoxic or hyperoxic modes A, B), or to the normoxic output channel 30″′ (in said normoxic mode C).

According to a possible embodiment, the hypoxic gas mixture, provided by the device 1 operated according to the hypoxic mode A, comprises an oxygen percentage comprised between 13%±1%-15%±1%, preferably of 14%±1%, and the hyperoxic gas mixture, provided by the device 1 operated according to the hyperoxic mode B, comprises an oxygen percentage comprised between 34%±1%-36%±1%, preferably of 35%±1%, and said normoxic gas mixture, provided by the device 1 operated according to the normoxic mode C, comprises an oxygen percentage comprised between 20%±1%-22%±1%, preferably of 21%±1%.

More in particular, in the hypoxic and hyperoxic modes A, B, the pressurized breathable air is supplied to the membrane tube assembly 2, which is commanded through proper value of the air pressure and/or of the flow rate, such that the desired composition of the permeate and/or retentate gas mixtures may be obtained:
a feed air pressure set at value comprised between 5 bar (g) and 6 bar (g), preferably equal to 5.5 bar (g) for the hypoxic mode “A” for exploiting a response flow rate of ~30 l/min of hypoxic air stream through the hypoxic outlet 2a depleted to down 13%±1%-15%±1%, preferably 14%±1% of O₂ titre (concentrate=retentate side flowing under pressure),
a feed air pressure set at value comprised between 8 bar (g) and 9 bar (g), preferably equal to 8.5 bar (g), for the mode “B” for collecting a response flow rate of ~23 l/min of hyperoxic air stream through the hyperoxic outlet 2b enriched by 34%±1%-36%±1%, preferably 35% O₂ titre concentrated in said air (=permeate side naturally flowing at atmospheric pressure).

In the normoxic mode C, the feed air pressure can be set by the means 7′, 7″ for controlling the pressure Pi at a value comprised between 8 bar (g) and 9 bar (g), preferably equal to 8.5 bar (g) for collecting a flow rate of ~35 l/min of normoxic air with 20%±1%-22%±1%, preferably 21%±1% nominal O₂ titre.

In the following, a method for operating the device 1 according to the invention, for alternatively providing hypoxic, hyperoxic and normoxic breathable gas mixtures, is described.

The method comprises a first step (a) of selecting the operating mode of the device 1 among said hypoxic mode A, hyperoxic mode B and normoxic mode C stored in the control unit 100.

According to a preferred embodiment, the operating mode of the device 1 can be switched from the hypoxic mode A to the normoxic mode C and then to the hyperoxic mode B: a user can select the operating mode by means of the user interface.

The method according to the invention further comprises the step (b) of feeding pressurized air to the means 81, 82, 83, 84; 11 for providing a breathable gas mixture, to the pressure regulation branch 32.

Once the device 1 is operated, the at least one source of pressurized air 3′, 3″ feeds pressurized air to the air supply line 30, which is fed to the means 81, 82, 83, 84; 11 for providing a breathable gas mixture having residual contaminants values complying with EU Pharmacopoeia.

As above discussed, the breathable gas mixture supplied by the means 81, 82, 83, 84; 11 comply with the requirements of the EU Pharmacopoeia, i.e. a value of CO₂ contaminant within the “Aer medicinalis” residual value prescribed by the European Pharmacopoeia, i.e. <500 ppm, a value of CO contaminant to a residual value as prescribed by the “Aer medicinalis” of the EU Pharmacopoeia, i.e. <5 ppm, a value of residual H₂O of 67 ml/m3, a residual value of oil of 0.1 mg/m3.

The method according to the invention comprises a further step (c) of operating the means 7′, 7″ for controlling the pressure Pi of the breathable gas mixture for maintaining an output pressure level set by the control unit 100 according to the selected mode of operation A, B, C.

As above discussed, according to a possible embodiment, the means 7′, 7″ for controlling the pressure Pi are operated for obtaining different pressure values, according to the selected mode of operation A, B, C of the device 1.

In said hypoxic mode (A) the means 7′, 7″ for controlling the pressure Pi are operated by the control unit 100 for providing an air pressure value comprised between 5 bar (g) and 6 bar (g), preferably equal to 5.5 bar (g); in said hyperoxic mode (B) and in said normoxic mode (C) the means 7′, 7″ for controlling the pressure Pi are operated by the control unit 100 for providing an air pressure value comprised between 8 bar (g) and 9 bar (g), preferably equal to 8.5 bar (g).

The method according to the invention comprises a further step (d), wherein the breathable air, pressurized by the means 7′, 7″ for controlling the pressure Pi, is fed through valve means 106, to the pressurized air inlet 21 of the membrane tube assembly 2 (in said hypoxic or hyperoxic modes A, B), or to the normoxic output channel 30″′ (in said normoxic mode C).

As above discussed, according to a possible preferred embodiment, in said hypoxic mode (A) the hypoxic gas mixture supplied by the membrane tube assembly 2 comprises an oxygen percentage comprised between 13%±1%-15%±1%, preferably of 14%±1%, and
in said hyperoxic mode (B) the hyperoxic gas mixture supplied by the membrane tube assembly 2 comprises an oxygen percentage comprised between 34%±1%-36%±1%preferably of 35%±1%, and
in said normoxic mode (C) the normoxic gas mixture supplied by the means 7′, 7″ for controlling the pressure Pi of the breathable gas mixture comprises an oxygen percentage comprised between 20%±1%-22%±1%, preferably of 21%±1%.

In further detail, the membrane tube assembly 2 is commanded through proper value of the air pressure and/or of the flow rate, such that the desired composition of the permeate and/or retentate gas mixtures may be obtained:
a feed air pressure set at value comprised between 5 bar (g) and 6 bar (g), preferably equal to 5.5 bar (g) for the hypoxic mode “A” for exploiting a response flow rate of ~30 l/min of hypoxic air stream through the hypoxic outlet 2a depleted to down 13%±1%-15%±1%, preferably 14%±1% of O₂ titre (concentrate=retentate side flowing under pressure),
a feed air pressure set at value comprised between 8 bar (g) and 9 bar (g), preferably equal to 8.5 bar (g), for the mode “B” for collecting a response flow rate of ~23 l/min of hyperoxic air stream through the hyperoxic outlet 2b enriched by 34%±1%-36%±1%, preferably 35%±1% O₂ titre concentrated in said air (=permeate side naturally flowing at atmospheric pressure).

In the normoxic mode C, the feed air pressure can be set by the means 7′, 7″ for controlling the pressure Pi at a value comprised between 8 bar (g) and 9 bar (g), preferably equal to 8.5 bar (g) for collecting a flow rate of ~35 l/min of normoxic air with 20%±1%-22%±1%, preferably 21%±1% nominal O₂ titre.

In the method according to the invention, in said hypoxic mode (A) a hypoxic gas mixture is supplied by the membrane tube assembly 2 through the hypoxic outlet 2a, to the hypoxic output channel 30′, and to the dispensing device 5 for a user.

Preferably, the hypoxic gas mixture is diverted through valve means 104 to the dispensing device 5 for a user.

In said hyperoxic mode (B) the hyperoxic gas mixture is supplied by the membrane tube assembly 2 through the hyperoxic outlet 2b, to the hyperoxic channel 30″, and to the dispensing device 5 for a user.

Preferably, the hyperoxic gas mixture is diverted through valve means 105 to the dispensing device 5 for a user.

In said normoxic mode (C) the normoxic gas mixture is supplied by the means 7′, 7″ for controlling the pressure Pi to the normoxic output channel 30″′, and to the dispensing device 5 for a user.

Preferably, the normoxic gas mixture is diverted through valve means 106 to the dispensing device 5 for a user.

According to a possible embodiment, wherein the air dryer module 11 comprises a regenerative inlet 11a fluidly connected through at least one hypoxic regenerative channel 31′, 31″ with the hypoxic output channel 30′, and wherein the at least one hypoxic regenerative channel 31′, 31″ comprises valve means 204, 205 which are operated in the hypoxic mode A or hyperoxic mode B for allowing the flow of hypoxic gas mixtures to the regenerative inlet 11a of the air dryer module 11 in order to regenerate the dryer module 11.

Furthermore, the regenerative inlet 11a is fluidly connected through a normoxic regenerative channel 31″′ with said normoxic output channel 30″′ also, for allowing the flow of normoxic gas mixtures to the regenerative inlet 11a of the air dryer module 11 in order to regenerate the dryer module 11 in the normoxic mode C.

According to a possible embodiment, the method further comprises the step of detecting at least one body parameter VPₘ by using monitoring means for monitoring at least one body parameter, preferably at least oxygen saturation level SpO₂ and/or a heart rate BPM and/or the concentration or partial pressure of carbon dioxide CO₂ in the gas mixtures.

For example, the means for monitoring the body parameters VPm can comprise an oximeter, and/or a capnograph.

For example, the body parameters VPₘ which can be monitored comprise oxygen saturation level (SpO₂) and/or a heart rate (BPM).

Following the detection of the least one body parameter VPₘ, the method comprises the step of directly or indirectly calculating the oxygen saturation level SpO₂ from the detected body parameter VPm and comparing it with a maximum reference level SpO₂ r,max and with minimum reference level SpO_{2 r,min}.

According to a possible embodiment, the maximum reference level SpO_{2r,max} and with minimum reference level SpO_{2 r,min} can be stored in the control unit 100, and the measured value of oxygen saturation level SpO₂ is compared with said maximum and minimum level.

Preferably, the maximum reference level of oxygen saturation SpO_{2 r,max} is a percentage value comprised between 98% and 100%, and the minimum reference level of oxygen saturation SpO_{2 r,min} is settled case by case according to the specific protocol and the user's need, and preferably is a percentage value comprised between 84% and 86%.

The method further comprises the step of switching to the hyperoxic operating mode B of the device 1 for providing the hyperoxic gas mixture to the dispensing device 5 in case the oxygen saturation level SpO₂ is lower than the minimum reference level SpO_{2 r,min}, and to the hypoxic operating mode A of the device 1 for providing the hypoxic gas mixture to the dispensing device 5 in case in case said oxygen saturation level SpO₂ is higher than the maximum reference level SpO_{2 r,max}.

It has to be noted that the device 1 cannot directly switch between hypoxic mode A and hyperoxic mode B, or vice versa: the normoxic mode C is mandatory operated between the other two modes.

According to a possible embodiment, the device 1 can operate IHT protocol, i.e. it can be switched alternatively between hyperoxic mode and normoxic mode.

According to a further possible embodiment, the device 1 can operate IHHT protocol, i.e. it can be switched from hyperoxic mode B, to normoxic mode C and to hypoxic mode A. Then the device 1 can be switched again to normoxic mode C and then to hyperoxic mode B.

In a possible embodiment, the changeover of the operating mode in a IHHT protocol is triggered by the comparison of the measured value of the oxygen saturation level SpO₂ with the maximum and minimum reference levels SpO_{2 r,max} and SpO_{2 r,min} which can be stored in the control unit 100.

In particular, in a possible embodiment, when the user is breathing a hyperoxic gas mixture delivered to the dispensing device 5, the SpO₂ value is normally comprised between 98-100%, i.e. it is approximatively at maximum reference level of oxygen saturation SpO_{2 r,max}.

This condition wherein the oxygen saturation level SpO₂ reaches said maximum reference level SpO_{2 r,max} determines the changeover of operating mode.

This results in that the device 1 is operated for delivering a main portion of normoxic gas mixture (according to the normoxic mode C) to the dispensing device 5, and for delivering a portion of the normoxic gas mixture to the regenerative inlet 11a of the air dryer module 11 for regeneration purpose.

Following this changeover, the user is breathing a normoxic gas mixture having 20%±1%-22%±1%, preferably of 21%±1% oxygen delivered through the dispensing device 5, and the measured SpO₂ value gradually falls.

Then, the operating mode is changed over to hypoxic mode A.

Following this changeover, the user is breathing a hypoxic gas mixture having 13%±1%-15%±1%, preferably of 14%±1% oxygen delivered through the dispensing device 5, and the measured SpO₂ value gradually falls.

This condition wherein the oxygen saturation level SpO₂ reaches said minimum reference level SpO_{2r,min} determines the changeover of the operated valve means.

This results in that the device 1 is operated for delivering again the main portion of normoxic gas mixture to dispensing device 5, such that the measured SpO₂ value gradually rises back, and a portion of the normoxic gas mixture to the regenerative inlet 11a of the air dryer module 11 for regeneration purpose according to operating mode C.

Then the device 1 is operated according to operating mode B for delivering the hyperoxic gas mixture to dispensing device 5, and for delivering the hypoxic gas mixture to the regenerative inlet 11a of the air dryer module 11 for regeneration purpose.

Following this change of the operating valve, the user is breathing a hyperoxic gas mixture having 34%±1%-36%±1%, preferably of 35%±1% oxygen delivered to said dispensing device 5, and the measured SpO₂ value gradually rises back. As the oxygen enriched air is breathed by the user, the SpO₂ will quickly return to 98-100%. Some minutes of normoxic operating mode C is then followed by another changeover to a low oxygen cycle.

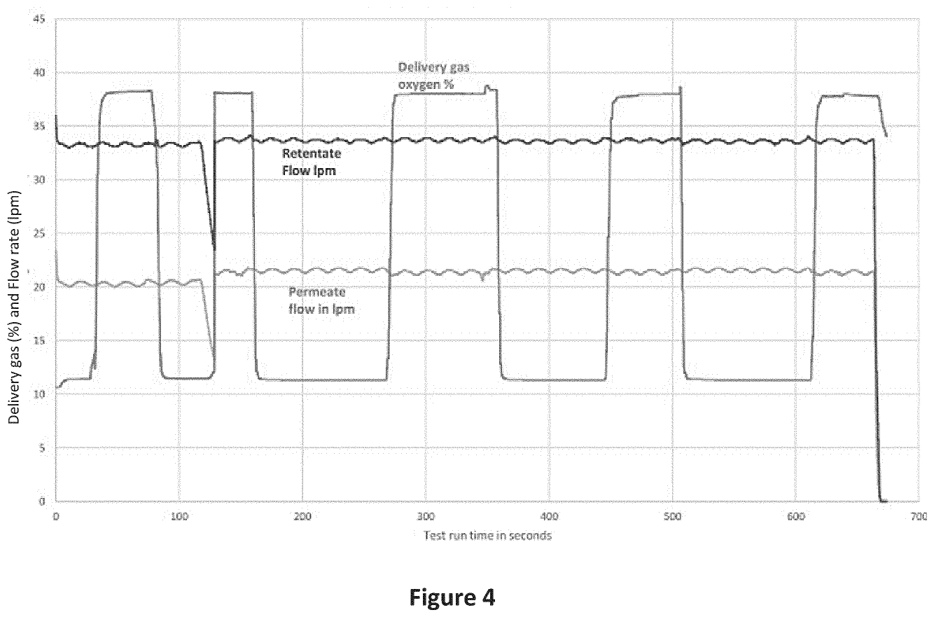

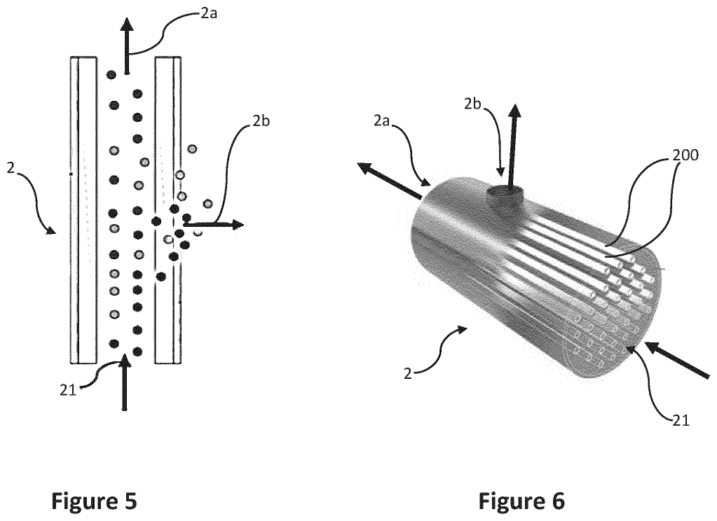

As shown in FIG. 4, this process provides a hysteresis: the effects of breathing an hypoxic gas mixture having reduced oxygen level, results in that the user's SpO₂ falls approximatively at minimum reference level of oxygen saturation SpO_{2 r,min}, for example through 85%.

Conversely, when the user is breathing a hyperoxic gas mixture having 34%±1%-36%±1% oxygen, delivered to said dispensing device 5, the SpO₂ value is normally comprised between 98-100%, i.e. it is approximatively at maximum reference level of oxygen saturation SpO_{2 r,max}.

1. A device (1) for producing hypoxic (mode A), hyperoxic (mode B) and normoxic (mode C) breathable gas mixtures, said device (1) comprising:
at least one source of pressurized air (3′, 3″) for feeding pressurized air to an air supply line (30),
at least one membrane tube assembly (2) for separating air into a retentate hypoxic gas mixture and a permeate hyperoxic gas mixture, said membrane tube assembly (2) comprising a pressurized air inlet (21), a hypoxic outlet (2a) fluidically connected to an hypoxic output channel (30′) which comprises a first valve (104), and a hyperoxic outlet (2b) fluidically connected to a hyperoxic output channel (30″) which comprises a second valve (105),
a gas supply device for supplying a breathable gas mixture (81, 82, 83, 84; 11), positioned along said air supply line (30) upstream of said membrane tube assembly (2), and comprising at least one air dryer module (11) and at least one filtration unit (81, 82, 83, 84),
a pressure controller (7′, 7″) for controlling the pressure (Pᵢ) of the pressurized breathable gas mixture, positioned along a pressure regulation branch (32) downstream of said gas supply device (81, 82, 83, 84; 11) and upstream of said membrane tube assembly (2), wherein said pressure controller (7′, 7″) is fluidically connected through a third valve (106) positioned along said pressure regulation branch (32), with said pressurized air inlet (21) of said membrane tube assembly (2) and with said normoxic output channel (30″′), and
a control unit (100) adapted to store operating parameters of said device (1) according to said hypoxic, hyperoxic and normoxic modes (A, B, C), connected at least to said pressure controller (7′, 7″), and connected at least to said first, second and third valves (104, 105, 106) for controlling the flow of hypoxic, hyperoxic or normoxic gas mixtures through the respective hypoxic, hyperoxic or normoxic output channels (30′, 30″, 30′″).

2. The device according to claim 1, wherein said hypoxic gas mixture has an oxygen percentage of 13%±1%-15%±1%, said hyperoxic gas mixture has an oxygen percentage of 34%±1%-36%±1%, and said normoxic gas mixture has an oxygen percentage of 20%±1%-22%±1%.

3. The device according to claim 1, further comprising a dispensing channel (51) and a dispensing device (5) for delivering said hypoxic, hyperoxic and normoxic breathable gas mixtures.

4. The device (1) according to claim 1, wherein said pressure controller (7′, 7″) is fluidically connected to said at least one source of pressurized air (3′, 3″) through a recirculation duct (34), for feeding back at least a portion of the pressurized breathable gas mixture to said source of pressurized air (3′, 3″).

5. The device (1) according to claim 1, wherein said control unit (100) is configured to operate said pressure controller (7′, 7″) such that:
in said hypoxic mode (A) the air pressure value is between 5 bar (g) and 6 bar (g), and
in said hyperoxic mode (B) and in said normoxic mode (C), the air pressure value is between 8 bar (g) and 9 bar (g),

6. The device (1) according to claim 1, wherein said air dryer module (11) comprises a regenerative inlet (11a) fluidically connected through at least one hypoxic regenerative channel (31′, 31″) with said hypoxic output channel (30′).

7. The device (1) according to claim 1, wherein said air dryer module (11) comprises a regenerative inlet (11a) fluidically connected through a normoxic regenerative channel (31″′) with said normoxic output channel (30′″).

8. The device (1) according to claim 6, further comprising at least one transducer (15, 16, 17) positioned along a dispensing channel (51) for controlling and operating at least further valves (204, 205) positioned along said at least one hypoxic regenerative channel (31′, 31″), and/or along a normoxic regenerative channel (31″′).

9. The device (1) according to claim 2, further comprising a flow rate controller (9′, 9″) that controls the flow rate of the breathable gas mixture provided by said device (1), positioned along said dispensing channel (51) upstream of said dispensing device (5).

10. The device (1) according to claim 1, wherein said at least one filtration unit (81, 82, 83, 84) for filtering the air drawn by said source of pressurized air (3′, 3″) comprises a filter cartridge (81) for reducing the value of the carbon dioxide (CO₂) contaminant in said air below 500 ppm, and/or a filter cartridge (82) for reducing the carbon monoxide (CO) contaminant in said air below 5 ppm, and/or a coalescer filter (83) for capturing H₂O droplets and for removing solid dust particles having an average dimension of at least 0.1 μm, and/or a dust filter (84) for removing solid dust particles having an average dimension of at least 0.1 μm, from said air.

11. The device (1) according to claim 1, wherein said hyperoxic output channel (30″) comprises a venting branch (33) for venting hyperoxic gas mixture when the device (1) is operated in said hypoxic mode (A).

12. Method for operating said device (1) according to claim 1, for alternatively providing hypoxic, hyperoxic and normoxic breathable gas mixtures, wherein said method comprises the following steps:
(a) selecting an operating mode of the device (1) among said hypoxic mode (A), hyperoxic mode (B) and normoxic mode (C) stored in said control unit (100),
(b) feeding pressurized air to said gas supply device (81, 82, 83, 84; 11) to provide a flow of breathable gas mixture to said pressure regulation branch;
(c) operating said pressure controller (7′, 7″) to control the pressure (Pi) of the breathable gas mixture along said pressure regulation branch (32) to maintain an output pressure level set by the control unit (100) according to the selected mode of operation (A, B, C), (d) feeding said pressurized breathable gas mixture to said pressurized air inlet (21) of said membrane tube assembly (2) in said hypoxic mode (A) or hyperoxic mode (B) of operation of said device (1), or to said normoxic output channel (30″′) in said normoxic mode (C) of operation of said device (1), wherein:
in said hypoxic mode (A), a hypoxic gas mixture is supplied by said membrane tube assembly (2) through said hypoxic outlet (2a), to said hypoxic channel (30′), and to said dispensing device (5);
in said hyperoxic mode (B) a hyperoxic gas mixture is supplied by said membrane tube assembly (2) through said hyperoxic outlet (2b), to said hyperoxic channel (30″), and to said dispensing device (5); and
in said normoxic mode (C) a normoxic gas mixture is supplied by said pressure controller (7′, 7″) to said normoxic channel (30″′), and to said dispensing device (5).

13. The method according to claim 12, wherein at least a portion of the pressurized breathable gas mixture is recirculated back through a recirculation duct (34) to said source of pressurized air (3′, 3″), in order to reduce the pressure value of the pressurized breathable gas mixture according to the selected mode of operation (A, B, C) of the device (1).

14. The method according to claim 13, wherein in said hypoxic mode (A) said pressure controller (7′, 7″) is operated to provide an outlet pressure value comprised between 5 bar (g) and 6 bar (g);
in said hyperoxic mode (B) and in said normoxic mode (C) said pressure controller (7′, 7″) is operated to provide an air pressure value between 8 bar (g) and 9 bar (g).

15. The method according to claim 12, wherein
in said hypoxic mode (A) said hypoxic gas mixture supplied by the membrane tube assembly (2) has an oxygen percentage of 13%±1%-15%±1%, and
in said hyperoxic mode (B) said hyperoxic gas mixture supplied by the membrane tube assembly (2) has an oxygen percentage of 34%±1%-36%±1%, and
in said normoxic mode (C) said normoxic gas mixture supplied by said pressure controller (7′, 7″) has an oxygen percentage of 20%±1%-22%±1%,

16. The method according to claim 12, further comprising a step of regenerating said air dryer module (11) by feeding a flow of hypoxic gas mixture to said air dryer module (11).

17. The method according to claim 12, further comprising a step of regenerating said air dryer module (11) by feeding a flow of normoxic gas mixtures to said air dryer module (11).

18. The device according to claim 1, wherein said hypoxic gas mixture comprises an oxygen percentage of 14±1%, said hyperoxic gas mixture comprises an oxygen percentage of 35%±1%, and said normoxic gas mixture comprises an oxygen percentage of 21%±1%.

19. The method according to claim 12, wherein:
in said hypoxic mode (A) said pressure controller (7′, 7″) is operated to provide an outlet pressure value equal to 5.5 bar (g); and
in said hyperoxic mode (B) and in said normoxic mode (C) said pressure controller (7′, 7″) is operated to provide an air pressure value equal to 8.5 bar (g).
